(19) United States
(12) Patent Application Publication
(10) Pub. No.: US 2008/0004439 A1
(43) Pub. Date: Jan. 3, 2008

(54) Sucrose-6-Ester Chlorination by Co-Addition of Chlorination Reagent

(76) Inventors: Rakesh Ratnam, Maharashtra (IN); Sundeep Aurora, Maharashtra (IN)

Correspondence Address: NATH & ASSOCIATES, 112 South West Street, Alexandria, VA, 22314, US

(21) Appl. No.: 11/794,495

(22) PCT Filed: Dec. 23, 2005
(86) PCT No.: PCT/IN05/00434

§ 371 Date: Jun. 29, 2007

(30) Foreign Application Priority Data
Jan. 3, 2005 (IN) 1/MUM/2005

(51) Int. Cl. C07H 1/00

(52) U.S. Cl. 536/124

(57) ABSTRACT

An improved process for chlorination is described wherein a solution of chlorinating agent and solution of sucrose-6-ester are mixed together by co-addition to a reaction vessel, the addition of both the reactants starting and completing substantially at the same time. The product of chlorinated sucrose is further extracted in organic solvents and deacylated to produce the high intensity sweetener product 4, 1′, 6′ trichlorogalactosucrose.

### TECHNICAL FIELD

The present invention relates to a process and a novel strategy of chlorination in the process for synthesis of chlorinated sucrose, 1′-6′-Dichloro-1′-6′-DIDEOXY-β-Fructofuranasyl-4-chloro-4-deoxy-galactopyranoside (TGS).

### BACKGROUND OF THE INVENTION

Chlorinated sucrose preparation is a challenging process due to the need of chlorination in selective less reactive positions in sucrose molecule in competition with more reactive positions. Generally, this objective is achieved by a procedure which involves essentially protecting the most reactive primary 6-hydroxy group in the pyranose ring of sugar molecule by converting it to ether aromatic or aliphatic esters or orthoesters, and the protected sucrose is then chlorinated in the desired positions 1′, 6′ and 4 to give the acetyl derivative of the product, which is then deacylated to give the desired product 1′-6′-Dichloro-1′-6′-DIDEOXY-β-Frutofuranasyl-4-chloro-4-deoxy-galactopyranoside i.e. 4,1′, 6′ trichierogalactosucrose (TGS).

Strategies of prior art methods of production described by Mufti et al. (1983) in U.S. Pat. No. 4,380,476 are based on chlorinating sucrose-6-acetate by a reagent capable of chlorinating at 1′, 4 and 6′ positions to form a trichloro derivative. For achieving chlorination, solution of sucrose-6-acetate is added to the solution of chlorinating reagent. This is followed by a schedule of regulated heating at various temperatures. The reactants, i.e. by addition of at least seven molar equivalents of a chlorinating reagent such as phosgene directly in to the solution of sucrose-6-acetate in dimethylformamide (DMF) under controlled conditions. i.e. by reversal of sequence of addition and the chlorinating reagent being added directly rather than after its conversion to Vilsmeier reagent. Examples have been given by Walkup et al. (1990) for this reversed sequence or addition using phosgene, thionyl chloride and phosgene iminium chloride as chlorination reagents. No examples are given, however, for the reversal of addition sequence for other chlorinating reagents, including phosphorus pentachloride, oxalyl chloride; rather examples are given for these chlorinating reagents, but with the sequence in which a chlorinating reagent was added by Mufti et al (1998). Rest of the aspects of Walkup et al. (1990) including reaction conditions for chlorination, deacetylation are disclosed by Mufti et al. (1983) already in fact, with phosphorus pentachloride as chlorinating reagent, inventors of this specification consistently received inferior yields by method of addition disclosed by Walkup et al (1990) than even the conventional method i.e. method of addition disclosed by Mufti et al. (1983). It seems that improvement in the efficiency of the method disclosed by Walkup et al. (1990) over conventional method is limited to use of phosgene, phosgene iminium chloride and oxalyl chloride as chlorinating agents.

### THE PRIOR ART

Mufti et al., in U.S. Pat. No. 4,380,476, has described the preparation of chlorinated sucrose-6-esters by the Vilsmeier Haack reagent and also sulphuryl chloride. Likewise Rathbone et al, in U.S. Pat. No. 4,617,269, has disclosed in the experiments relating to such chlorination steps.

Further Rathbone, in U.S. Pat. No. 4,324,888, has described the reaction of a reducing sugar and vilsmeier reagent to obtain the mono chlorinated sugar derivatives, This parent discusses the direct replacement of hydroxyl groups by chlorine using various reagents, including chloroformimiminium chloride Use of Vilsmeier reagent in preparation of halo sugars has been elaborately discussed in Walter A. Szarek, “Deoxyhalogeno Sugars”, ir. Advances in Carbohydrate Chemistry & Biochemistry, 28,225-307 (1973), at 230-259. Also references from Hanessian et al., “A New Synthesis of Chlorodeoxy-sugars”, Chem. Commun., 1967, 1152-1155, describe the N,N-dimethylchloroformiminium chloride in the synthesis of chlorodeoxy sugars., (R. L. Whistler and A. K. M. Anisuzzaman in “Methods in Carbohydrate Chemistry”, Vol. VIII, R. L. Whistler and J. N. BeMiller, Eds., Academic Press, New York, 1980, pp. 227-231)., Eilingsfeld et al., Angew. Chem. 72 (22), 836-845 (1960),

### SUMMARY OF INVENTION

This specification discloses a method of contact of reactants for chlorination of sucrose-6-acetate which gives better yields and lesser formation of difficult-to-remove impurities and, hence, a cleaner product than the methods known so far. An improved and highly efficient way of producing chlorinated sucrose derivatives and recovering them from reaction mixture is described. This is accomplished by simultaneous addition i.e. co-addition, of acid chlorides like POCl₃ or PCl₅ and substrate to be chlorinated to the reaction vessel solvent. Alternatively, even the prepared Vilsmeier-Haack reagent (Vilsmeier) may be added simultaneously to the substrate to be chlorinated to the reaction solvent

The yields of the chlorinated sucrose derivatives obtained by the above process are better when compared to the methods of chlorination by conventional method (described by Mufti et al. 1983) and the method described by Walkup et al. (1990) using acid chlorides including POCl₃ or PCL₅.

It has also been seen that method of this invention produced product with lesser amount of tetrachloro impurities than the product produced by the method of Walkup et al. (1990).

### DETAILED DESCRIPTION OF INVENTION

The method for synthesis of chlorinated sucrose according to an embodiment of the present invention involves following steps:

A solution of desired molar concentration of the chlorinating agent and solution of the substrate to be chlorinated are added to and mixed simultaneously in a reaction flask containing an excess amount of tertiary amide; conducting the addition at a controlled reduced temperature as described below, followed by heating at various levels of elevated temperature for a regulated period of time. The chlorinated mass is then cooled to 70-85° C. and neutralized with a solution containing hydroxides of alkali metals such as sodium, potassium, etc., or alkali earth metals such as calcium, barium, etc., wherein the efficiency of chlorination reaction is found to be very good by this new route.

The addition of reactants for the purpose of mixing needs to be a well regulated flow. According to an embodiment, the regulation of flow may be done by drop-wise addition of the reactants. The regulation of flow may also include, but not limiting to addition of small stream of the reactants and the like.

As a variation of this method, it is also possible to visualize no prior addition of excess DMF in the reaction flask and all the excess DMF that is envisaged to be required participate in the reaction is added either to sucrose-6-acetate solution or to chlorinating reagent solution or distributed amongst both. The option to add DMF to the reaction flask prior to the co-addition of the chlorinating reagent and sucrose-6-acetate solution in DMF depends upon whether an excess of DMF is incorporated in one or both of the reactants a priori or not. If it has been added, there is no need of prior addition of excess of DMF to the reaction vessel. The amount of DMF should be enough to keep the reactants as well as the products of the reaction in solution.

Method of Vilsmeier preparation using chlorinating agent such as POCl₃, PCl₅, etc., and tertiary amide such as dimethylformamide and further contacting of the substrate to be chlorinated with the prepared Vilsmeier is very important. Also the temperature during the Vilsmeier preparation and addition of substrate plays a vital role. Further, the reaction mass is heated to elevated temperature gradually to various levels to achieve desired levels of chlorination.

The substrate to be chlorinated usually is a sucrose ester derivative having ester group at 6^{th} position of pyranose ring of sugar molecule including sucrose-6-acetate or sucrose-6-benzoate. The substrate is dissolved in a tertiary amide solvent free from moisture, preferably dimethylformamide. The chlorinating agent such as POCl₃, PCl₅, etc., or Vilsmeier reagent prepared from same, dissolved in DMF in desired molar proportion is added simultaneously along with the sucrose derivative dissolved in a tertiary amide such as dimethylformamide drop-wise to a reaction flask containing volume in excess of the said tertiary amide. The addition is carried out at a temperature between −30° C. to +20° C.; more preferably between −50° C. to 0° C.

After the complete addition of the chlorinating agent and the substrate, the reaction mass is heated to about 85° C. for 1-3 hours, preferably 1 hr., then to about 100° C. for 6-10 hrs., preferably 8 hrs and further heated to about 110-120 , preferably 114-115° C. and held for 1-3 hrs, preferably to 1.5 hours. The chlorinated mass is then cooled to 70-85° C. and neutralized with a solution containing hydroxides of alkali metals such as sodium, potassium, etc., or alkali earth metals such as calcium, barium, etc. So far, yields up to 60% have been successfully obtained in this way and further fine-tuning and improvement is in progress.

In simplest embodiment of this invention, the solution of sucrose-6-acetate taken for chlorination could be derived by dissolving sucrose-6-acetate, pure or of various degrees of purity, in a tertiary amide, preferably dimethylformamide. At the same time, it is also possible to start with a reaction mixture of a process stream derived from manufacture of TGS or 6-acetyl-TGS and chlorination of such mixture by process described in this specification is also an example of embodiment of this invention. Such process streams are generated in processes aiming at production of sucrose-6-acetate itself, 6-acetyl-TGS or TGS including but not limited to patents described by Mufti et al. (1983) in U.S. Pat. No. 4,380,476, Simpson (1989) in U.S. Pat. No. 4,889,928, Neiditch, et al. (1991) in U.S. Pat. No. 5,023,329, Walkup et al. (1992) in U.S. Pat. No. 5,089,608, Dordick et al. (1992) in U.S. Pat. No. 5,128,248, Khan et al. (1995) in U.S. Pat. No. 5,440,026, Sankey (1995) in U.S. Pat. No. 5,449,772, Sankey et al. (1995) in U.S. Pat. No. 5,470,969 and by Navia et al (1996) in U.S. Pat. No. 5,530,106.

Adaptation of co-addition method for chlorination of pentacetate of sucrose is also possible and is also covered within the scope of this specification as an embodiment of this invention

An adaptation of this invention may also include use of Sucrose 6,4′-dicarboxylic esters described by Dordick et al. (1993) in U.S. Pat. No. 5,270,460 for chlorination by co-addition which shall also be an embodiment the invention disclosed in this specification.

Examples given in the following serve to illustrate manner of performing the invention without limiting the scope of reaction conditions for the purpose of optimizing the yield or for any other purpose. Any reasonable variation of the process described, modifications obvious to a person skilled in the art and analogous processes with analogous reactants are included within the scope of this specification.

Anything mentioned in singular applies to its plural also e.g. “an organic solvent” includes any and every organic solvent that is applicable to the context and more than one or a combination of organic solvents applicable in the context.

### EXAMPLE 1

Chlorination of Sucrose-6-Acetate by Co-addition

85 g of crude sucrose-6-acetate (82% pure, 0.18 moles) dissolved in 300 ml of dimethylformamide was taken for the chlorination reaction. 500 ml of Dimethylformamide was taken in a reaction flask and was cooled to −5° C. The reaction flask was fitted with two addition funnels. POCl₃ 103 ml (1.1 moles) was taken in one of the addition funnel and the sucrose-6-acetate solution in the other. The addition of POCl₃ and the sucrose-6-acetate solution was started and the temperature was controlled below 0° C. The rate of addition of both the solutions was adjusted in such a way so that addition is completed substantially at the same time.

The reaction mass was then allowed to come to room temperature and heated to 85° C. and maintained for 1.0 hr. Then it was further heated to 100° C. and maintained for 8 hours and further heated to 115° C. and maintained for 1.5 hrs with frequent TLC analysis. The reaction mass was then neutralized with calcium hydroxide slurry and the pH was adjusted to 7.5.

The product, 6 acetyl 4,1′, 6′ trichlorogalactosucrbse, yield from sucrose-6-acetate stage in the neutralized mass was found to be 35.8%.

The HPLC analysis was carried out in C18 column and the mobile phase used was 85:15 of Water Acetonitrile. The identity of the product was confirmed with comparison to the USP standard TGS.

The reaction mass containing 6-acetyl TGS was then passed through ATFD. The DMF free solids obtained were then dissolved in 1:4 times of water and then extracted into 1:1 times v/v of ethyl acetate. The ethyl acetate was then distilled off to obtain a syrup which was loaded into Silanized silica gel. The pure fractions of 6-acetyl TGS were collected and pooled, deacetylated and the product TGS crystallized. Recovery of TGS with respect to sucrose-6-acetate taken for above reaction was 30%

### EXAMPLE 2

Comparison of Conventional Method of Chlorination and Co-addition

Solution of sucrose-6-acetate (85 g of 82% purity) was dissolved in DMF (300 ml). POCL3 was taken in a molar proportion of 4 moles to 10 moles.

In conventional method, desired quantity of POCl₃ (to give 4 to 10 molar equivalents with respect to sucrose-6-acetate taken for the reaction) was added drop-wise to DMF under stirring to the reaction flask. The Vilsmeier formation was indicated by orange coloured solution in the flask. After complete addition of POCl₃ to DMF, the solution of sucrose-6-acetate in DMF was added drop-wise to the prepared Vilsmeier below 5° C.

In method of this invention, desired moles of POCL₃ (4 to 10 molar equivalents) were taken in a funnel and sucrose-6-acetate in DMF was taken in another funnel and added to a reaction vessel containing excess of DMF and temperature was controlled below 0° C. The addition was regulated such that addition of both the solutions ended substantially at the same time.

In another method, desired amount of POCl₃ (to give 4 to 10 molar equivalents with respect to sucrose-6-acetate taken for the reaction) was added drop-wise to DMF under stirring to the reaction flask. The temperature was controlled below 5° C. The Vilsmeier formation was indicated by orange coloured solution in the flask. Sucrose-6-acetate in DMF was taken in another flask and to it an addition funnel was fitted. The prepared vilsmeier from the reaction flask was added taken in the additional funnel and was added drop-wise to sucrose-6-acetate solution. The temperature was controlled below 5° C.

The reaction mass of all three methods of addition were then allowed to come to room temperature and heated over 25 min to 60° C. and held at this temperature with stirring under argon for 5 min. The solution was heated to 83° C. over 15 minutes and held at this temperature for 65 min. The reaction temperature was then increased to 115° C. over about 20 min and held at this temperature for 187 min frequent TLC analysis. The reaction mass was then neutralized with calcium hydroxide slurry and the pH was adjusted to 7.5.

Then the reaction mass containing 6-acetyl TGS from each of the reaction was passed through ATFD. The respective solids obtained after ATFD was dissolved in 1:3 times of water and then extracted into 1:3 volumes of ethyl acetate. The ethyl acetate was stripped off and the syrup obtained was taken for purification in silanized silica column. The pure fractions of 6-acetyl TGS obtained was concentrated, deacetylated and crystallized by suitable methods.

Yields achieved from above experiment are given in Table 1 and amount of tetrachloro impurities obtained in the product TGS are given in Table 2 below.

TABLE 1
Yields of TGS achieved by three different sequences of addition
of reagents of chlorination reaction involving solutions of
chlorinating reagent and solution of sucrose-6-acetate.

| | Sequential | | |
|---|---|---|---|
| | addition of | Co addition of | Addition of |
| | chlorinating | chlorinating | vilsmeier to |
| Molar ratio of | agent and | agent and | sucrose-6- |
| chlorinating | sucrose-6- | sucrose-6- | acetate |
| agent to | ester (TGS | ester (TGS | solution (TGS |
| substrate | % yield) | % yield) | % yield) |
| 4.0 moles | 12% | 20% | 10% |
| 5.0 moles | 15.6% | 26% | 13% |
| 6.0 moles | 17.4% | 36% | 12.3% |
| 7.0 moles | 21.6% | 37.2% | 16.6% |
| 8.0 moles | 23.0% | 38.2% | 18.5% |
| 9.0 moles | 23.6% | 38.6% | 20.8% |
| 10 moles | 23.4% | 38.5% | 20.6% |

TABLE 2
Concentration of tetrachloro impurities of TGS formed in a
chlorination reaction by three different sequences of addition
of reagents of chlorination reaction involving solutions of
chlorinating reagent and solution of sucrose-6-acetate

| | Sequential | | |
|---|---|---|---|
| | addition of | Co addition of | Addition of |
| | chlorinating | chlorinating | vilsmeier to |
| Molar ratio of | agent and | agent and | sucrose-6- |
| chlorinating | sucrose-6- | sucrose-6- | acetate |
| agent to | ester (TGS | ester (TGS | solution (TGS |
| substrate | % yield) | % yield) | % yield) |
| 4.0 moles | 8% | 6% | 7.2% |
| 5.0 moles | 10.9% | 7.8% | 10.4% |
| 6.0 moles | 13% | 9.6% | 10.3% |
| 7.0 moles | 15.6% | 11.19% | 13.6% |
| 8.0 moles | 19.4% | 13.6% | 19.5% |
| 9.0 moles | 22.35% | 15.8% | 23.8% |
| 10 moles | 26.14% | 18.9% | 25.6% |

### EXAMPLE 3

Chlorination of Sucrose-6-Benzoate by Co-addition

Crude sucrose-6-benzoate, 5 kg, dissolved in 21.50 L of dimethylformamide was taken for the chlorination reaction. Dimethylformamide, 36 L, was taken in a reactor and was cooled to −5° C. POCl₃, 5.2 L, was taken in one of the dozing vessel and the sucrose-6-benzoate solution in the other which was connected to the reactor. The addition of POCl₃ and the sucrose-6-benzoate solution was started and the temperature was controlled below 0° C. The rate of addition of both the solutions was adjusted in such a way so that addition is completed substantially at the same time.

The reaction mass was then allowed to room temperature and heated to 85° C. and maintained for 1.0 hr. Then it was further heated to 120° C. and maintained for 3½ hours with frequent TLC analysis. The reaction mass was then neutralized with calcium hydroxide slurry and the pH was adjusted to 7.5.

The product, 6 benzoyl 4,1′, 6′ trichlorogalactosucrose yield from sucrose-6-benzoate stage in the neutralized mass was found to be 36%.

### EXAMPLE 4

Comparison of TGS Yield by Co-addition and Conventional Sequential Addition

Dimethylformamide, 270 g, was taken in a reaction flask and was cooled to 10° C. PCl₅, 266 g, was added to the flask with constant stirring. The Vilsmeier reagent was allowed to form, this was seen by the solids falling out as crystals. Along with the crystals a orange to brown colored solution was formed due to the second Vilsmeier formation from POCl₃ liberated from the PCl₅ reaction.

The brown colored solution was separated from the Vilsmeier salt formed. The Vilsmeier salt was washed with excess DMF. The Vilsmeier DMF slurry was taken for chlorination reaction.

200 ml of DMF was taken in a reaction flask and was cooled to 5° C. The flask was fitted with 2 addition funnels, Vilsmeier slurry was taken in one of them and 100 g of crude 6-O-acetylsucrose dissolved in 320 ml DMF was taken in other.

The addition of the Vilsmeier slurry and the 6-O-acetylsucrose solution was started and the temperature was controlled below 15° C. The rate of both additions was adjusted in such a way so that addition is completed substantially at the same time.

The reaction mass was then allowed to room temperature and heated to 85° C. and maintained for 1.0 hr. Then it was further heated to 120° C. and maintained for 3½ hours with frequent TLC analysis. The reaction mass was then quenched with calcium hydroxide slurry and the pH was adjusted to 7.5.

The product, 6 acetyl 4,1′, 6′ trichlorogalactosucrose yield from 6-O-acetylsucrose stage in the neutralized mass was found to be 55%

The comparison of TGS yield by co-addition and conventional sequential addition. Results are shown in the table 3 with respect to PCl₅ as chlorinating agent

TABLE 3
Comparative yields obtained by co-addition

| | Sequential addition of | |
|---|---|---|
| Molar ratio of | chlorinating agent and | Co-addition of |
| chlorinating | sucrose-6-ester | chlorinating agent |
| agent to substrate | (TGS % yield) | and sucrose-6-ester |
| 4.0 moles | 16% | 20.2% |
| 5.0 moles | 18.4% | 26.4% |
| 6.0 moles | 22.6% | 32.8% |
| 7.0 moles | 34.6% | 46.2% |
| 8.0 moles | 40.6% | 55.2% |
| 9.0 moles | 43.2% | 58.6% |
| 10 moles | 45.2% | 60.0% |

1. A process of chlorination of sucrose-6-ester dissolved in a solvent by bringing its solution in contact with a chlorinating reagent by co-addition comprising adding both the solutions in a regulated stream of flow at the same time into a reaction vessel wherein the addition of both the streams starts and ends substantially at the same time and, preferably, during the addition, temperature of the reaction is regulated within a range of −30 to +20° C., preferably within a range of −5° C. to 0° C.

2. A process of claim 1 wherein the said sucrose-6-ester is sucrose-6-acetate or sucrose-6-benzoate derived:
a. as a solution made from the solids containing sucrose-6-acetate or sucrose-6-benzoate in substantially pure form, or
b. as a process stream from a process of production of sucrose-6-benzoate, sucrose-6-acetate, 6-acetyl-1′-6′-Dichloro-1′-6′-Dideoxy-β-Fructofuranasyl-4-chloro-4-deoxy-galactopyranoside (6-acetyl-TGS) or TGS.

3. A process of claim 1 wherein the said solvent in which sucrose-6-acetate or sucrose-6-benzoate is dissolved is a tertiary amide, preferably dimethylformamide.

4. A process of claim 3 wherein the said chlorinating agent comprise:
a. one or more of acid chlorides including phosphorus oxychloride, phosphorus pentachloride, thionyl chloride, oxalyl chloride, phosgene iminium chloride, sulphuryl chloride, phosgene; or
b. a Vilsmeier reagent derived from an acid chloride including phosphorus oxychloride, phosphorus pentachloride, thionyl chloride, oxalyl chloride, phosgene iminium chloride, sulphuryl chloride, phosgene.

5. A process of claim 4 wherein the said regulated flow includes drop-wise addition of the solutions of sucrose-6-acetate or sucrose-6-benzoate and chlorinating reagent into a reaction vessel, addition of both solutions starting and ending substantially at the same time.

6. A process of claim 3 wherein an excess of dimethylformamide is added to the said reaction vessel before co-addition of the solution of sucrose-6-acetate or sucrose-6-benzoate and the solution of chlorinating reagent begins.

7. A process of claim 1 further comprising the steps of:
a. Cooling the reaction mass to room temperature, s
b. heating the reaction mass to about 70 to 90° C., preferably to 85° C. and maintaining the temperature for a period of time sufficient to maximum achievable monochlorination, preferably for about 1.0 hr,
c. further heating to about 90 to 110° C., preferably to 100° C. and maintaining for period of time sufficient for maximum achievable dichlorination, preferably for about 8 hours, and
d. further heating to about 115 to 125° C., preferably for 115° C. and maintained for a period sufficient to achieve complete trichlorination, preferably for about 1.5 hrs.

8. A process of claim 1 further comprising steps of
a. Cooling the reaction mass to room temperature,
b. Heating to about 60° C. and maintaining at this temperature with stirring under an inert gas including argon, nitrogen initially for about 5 min. and then preferably for about 15 to 30 minutes, more preferably for about 25 min,
c. further heating the solution over a period of about 15 minutes to about 75 to 85° C., preferably to about 83° C. and maintaining at that temperature for a period of time sufficient to achieve complete monochlorination as well as initiation of dichlorination, preferably for a period of about 60 to 70 minutes, more preferably for about 65 min. and
d. further increasing the temperature over a period of time of about 20 min to around 115° C., maintaining at this temperature for a period of time sufficient to achieve completion of dichlorination and further conversion into a trichloro derivative, preferably for a period of about 150 to 200 minutes, more preferably for about 190 min.

9. A process of claim 1 further comprising a step of recovering 6-acetyl-TGS formed after chlorination from the reaction mixture by extracting in an organic solvent.

10. A process of claim 9, wherein the organic solvent includes methyl tertiary butyl ether or ethyl acetate.

11. A process of claim 2 wherein the said solvent in which sucrose-6-acetate or sucrose-6-benzoate is dissolved is a tertiary amide, preferably dimethylformamide.

12. A process of claim 5 wherein an excess of dimethylformamide is added to the said reaction vessel before co-addition of the solution of sucrose-6-acetate or sucrose-6-benzoate and the solution of chlorinating reagent begins.
